(19) United States
(12) Patent Application Publication
(10) Pub. No.: US 2026/0022348 A1
(43) Pub. Date: Jan. 22, 2026

(54) Tissue slice cryopreservation

(71) Applicant: Regents of the University of Minnesota, Minneapolis, MN (US)

(72) Inventors: Srivasupradha Ramesh, St. Paul, MN (US); Joseph Sushil Rao, Minneapolis, MN (US); Bat-Erdene Namsrai, Plymouth, MN (US); Michael L. Etheridge, Maple Grove, MN (US); Timothy L. Pruett, Minneapolis, MN (US); Erik B. Finger, Wayzata, MN (US); John C. Bischof, St. Paul, MN (US)

(21) Appl. No.: 19/275,293

(22) Filed: Jul. 21, 2025

Related U.S. Application Data
(60) Provisional application No. 63/674,116, filed on Jul. 22, 2024.

(51) Int. Cl. C12N 5/00

(52) U.S. Cl. CPC C12N 5/525; C12N 5/542; C12N 5/562

(57) ABSTRACT

Methods and materials for cryopreservation of precision-cut tissue slices, including precision-cut liver slices (PCLS), where the slices are vitrified. A cryoprotective solution comprising high amounts of ethylene glycol in combination with sucrose are used and high cooling rates greater than the critical cooling rate for avoidance of ice formation are used in a method which results in vitrification of the tissue slices without toxicity and with increased viability after rewarming. Methods and materials can be used for production of PCLS which can be stored at cryoprotective temperatures for extended periods of time.

### CROSS REFERENCE TO RELATED APPLICATIONS

This application claims priority to and the benefit of U.S. provisional patent application Ser. No. 63/674,116, filed on Jul. 22, 2024, the contents of which are incorporated herein in their entirety.

### STATEMENT OF FEDERALLY SPONSORED RESEARCH

This invention was made with government support under DK132211 awarded by the National Institutes of Health, and EEC1941543 awarded by the National Science Foundation. The government has certain rights in the invention.

### BACKGROUND

Reported methods to cryopreserve tissue slices have not been able to successfully vitrify and rewarm slices to show viability, function, tissue architecture and enzymatic activity comparable to controls. Reported methods have not shown the ability to culture slices for multiple days post cryopreservation showing viability, function, tissue architecture and enzymatic activity comparable to controls.

There are large failure rates associated with drug development due to lack of cryopreservation methods for human tissue to be able to use them to mimic in-vivo environments of the prior art.

The costs of bringing a new pharmaceutical to market ranges between $2.6B and $6.7B, when including capital and failure costs. The decrease in successful R&D outcomes within the biopharma industry poses challenges to the global healthcare ecosystem by increasing price pressure and extending timeframes to deliver life-saving treatments to patients.

Current in-vitro drug testing and discovery models are mostly animal-derived and cell-based, and they fail to accurately predict the toxicity and efficacy of drugs in humans, leading to failures during clinical trials (Vernetti et al. 2017). In vitro drug metabolic studies use less complex testbeds, which do not adequately capture the complex dynamics of the multicellular mechanisms of the human body (Imamura et al. 2015, Langhans, S.A. 2018). This pushes us toward an R&D crossroads where the need for accurate and efficient test beds for biomedical research is critical. The need for alternative and more effective preclinical testing models is emphasized by the recent announcement by the US National Institutes of Health that it will no longer award funding to new grant proposals solely relying on animal testing.

Hepatotoxicity is the most common cause of pharmaceutical failure and withdrawal from the market. Ideally, drug metabolism is studied in vivo, but this becomes prohibitive in terms of cost, accessibility of samples, and ethical concerns. Alternatively, precision cut liver slices (PCLS) can act as a well-characterized model. PCLS maintains in-vivo architecture, representative cell populations, and function of the whole organ. Since their introduction 30 years ago, PCLS studies have demonstrated the ability to mimic drug pathways in humans for drug efficacy and toxicity studies (Astashkina et al. 2012, Lake et al. 1993). PCLS can also be created from diseased livers, making them excellent in-vitro disease models for a wide range of biomedical research. Arguably, the biggest barrier to PCLS use is the lack of standardized protocols for preserving PCLS like those that exist for cells (Astashkina et al. 2012). Improving protocols for preserving, storing, and shipping PCLS would broaden access for a variety of applications. However, like organs, the viability of PCLS rapidly degrades within hours in cold storage. Under culture, PCLS can last for only a few days, but viability and function decline with time in culture (Paish, 2019). Similarly, successful cryopreservation of precision cut tissue slices from other organs, including kidney, lung, heart, brain, and others, would provide valuable test models for pharmacotoxicology, preclinical, and biomedical research.

Cryopreservation, or the storage of biosystems at ultralow temperatures, could enable the on-demand supply of PCLS for pharmacotoxicology and other biomedical applications. However, conventional freezing cryopreservation protocols using slow cooling in dilute concentrations of cryoprotectants (e.g. DMSO) have proven unreliable for PCLS. While slow freezing with ice is commonly employed in cell suspensions, it fails in complex tissues where ice formation disrupts cellular and tissue structures, severely compromising viability and function (de Graaf et al., 2007). Indeed, the best PCLS freezing protocols published have resulted in only 70% viability after 4 hours in culture (de Graaf et al., 2007).

### DESCRIPTION OF THE FIGURES

FIG. 1A-1B: An overview PCLS cryopreservation. FIG. 1A illustrates previous works in the cryopreservation of PCLS report cooling rates that would lead to damaging ice formation (de Graaf et al., 2007, Rypka et al., 2006, Fisher et al., 1991, de Kanter et al. 1995) and FIG. 1B is a schematic diagram of the steps in the process for vitrification and rewarming using the cryomesh. with CPA, cryopreservation agent.

FIG. 2A-2E: Vitrification characteristics of PCLS. FIG. 2A: The CPA loading and unloading profile was an initial three steps of EG followed by a final step of 40% (v/v) EG and 0.6M Sucrose. Post vitrification, we unloaded the CPA in six steps of decreasing sucrose concentration and a final carrier solution-only step. This protocol was also used for other CPA formulations utilizing the same steps relative to the final concentration. FIG. 2B: The COMSOL model estimated the total CPA concentration throughout the tissue following loading steps of 5, 10, and 15 minutes. Other step durations could be used. FIG. 2C: Representative thermometry of the PCLS taken during vitrification and rewarming on the cryomesh. Inset is a picture of the thermometry setup. FIG. 2D: Cooling and rewarming rates measured from thermometry. FIG. 2E: Visualization of ice in the PCLS. Control and VR are transparent, indicating no ice, and the frozen slice is opaque due to ice formation. CPA, cryoprotective agent; VR, vitrification and rewarming.

FIG. 3A-3C: Viability and tissue morphology directly post cryopreservation. FIG. 3A: (i-v) Acridine Orange/Propidium Iodide (AO/PI) staining where AO-stained cells appear green, and the PI-stained cells are red, indicating cell membrane compromise. (vi-x) H&E staining of the slice groups just post rewarming. FIG. 3B: The viability through membrane integrity was assessed by analyzing the number of dead cells in the given z-plane by assessing areas of individual cells and comparing the number of PI quenched cells to the total number of cells. FIG. 3C: Viability measure from AO/PI for each group. Levels of significance: ***, p=0.0001; ****, p<0.0001 (One-way ANOVA). AO, acridine orange; CPA, cryoprotective agent; FT, slow freczing and rapid thawing; H&E, hematoxylin and cosin; PI, propidium iodide; VR, vitrification and rewarming.

FIG. 4A-4D: Metabolic and functional assessments of liver slices over 3 days in culture. FIG. 4A: ATP assessment. FIG. 4B: Urea production. FIG. 4C: Albumin synthesis. FIG. 4D: ATP assessment of VR group that was stored at −150° C. for 60 days (t=60). Levels of significance: * p<0.05, ** p<0.005, *** p<0.0005, and **** p<0.00005 (Mann-Whitney U test for FIGS. 4A, 4B and 4C and Kruskal-Wallis test for FIG. 4D. CPA, cryoprotective agent; FT, slow freezing and rapid thawing; VR, vitrification and rewarming.

FIG. 5A-5D. Acute injury response of slices and quantified enzymatic activity. FIG. 5A: Acute evaluation of PCLS with AO/PI, H&E staining, and CYPIAI zonated activity. FIG. 5B: TUNEL staining after 24 h culture to evaluate apoptosis (or necrosis) in both groups. Hematoxylin staining (blue) shows all nuclei and DAB stain (brown) shows TUNEL positive nuclei. FIG. 5C: Quantification of TUNEL-positive cells (n=7). FIG. 5D: Rates of resorufin production (N=3, n=4 where N indicates biological replicates and n indicates number of slices from a single biological replicate). The level of significance is presented by compact letter display (Kruskal-Walli's test). AO, acridine orange; CYP1A1, Cytochrome P450 1A1; H&E, hematoxylin and cosin; PI, propidium iodide; VR, vitrification and rewarming; CV, central vein.

FIG. 6A-6B. Drug response of PCLS. FIG. 6A: ATP levels at the end of day 3 in culture of control and VR slices exposed to varying concentrations of Acetaminophen (APAP). FIG. 6B: Urea levels measured normalized to OmM concentration spanning a 3-day culture period on exposure to different APAP concentrations. Data are mean±standard deviation. Levels of significance are represented by compact letter display (Dunn's test). APAP, N-acetyl-para-aminophenol (acetaminophen), VR, vitrification, and rewarming.

FIG. 7 is a chart illustrating the result of the total protein analysis of the liver slices including control “C”, CPA, VR and TF groups.

FIG. 8 is a chart illustrating the functional viability of PCLS after exposure to modified, vitrifiable CPA formulations, including EG+Sucrose (40% (v/v) EG+0.6M Sucrose, in carrier solution), DEPS (20% (v/v) EG+20% (v/v) DMSO+5% (v/v) PG+0.3M Sucrose, in carrier solution), and PD (24.4% (v/v) PG+22.8% (v/v) DMSO, in carrier solution). CPA loading followed the same relative protocol described in FIG. 2A.

### DEFINITIONS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

The terms “at least one” and “one or more of” an element is used interchangeably and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The terms “comprises,” “comprising,” and variations thereof are to be construed as open ended—i.e., additional elements or steps are optional and may or may not be present.

Unless otherwise specified, temperatures referred to herein are based on atmospheric pressure (i.e., one atmosphere).

“Cryopreservation” as used herein relates to preservation of a biological sample/specimen at cryogenic temperatures. Cryopreservation includes cooling/freezing the biological sample below subzero temperatures to suspend metabolic/chemical activity which can provide long term storage of biomaterials. Cryopreservation of a biological sample may also include warming the biological sample to superzero temperatures to recover the function/activity of the biological sample.

“Cryogenic” or “cryogenic temperature” as used herein relates to a temperature below sub-zero. Cryogenic temperatures can be in the range from −80° C. (−112° F.) to absolute zero (−273° C. or −460° F.) but includes any effects below the freezing point of the sample/specimen.

“Cryogenic coolant” or “cryogenic substance” or “cryogenic fluid” as used herein relates to a substance that is at a cryogenic temperature, e.g., liquid nitrogen, slush nitrogen. “Cryogenic coolant” or “cryogenic substance” or “cryogenic fluid” are used interchangeably herein.

“Cryoprotective solution” or “CPA cocktail” as used herein relates to a solution that includes one or more cryoprotective agents (CPA). Cryoprotective solution may be referred to as a “CPA solution” or a “CPA cocktail” or more generally “CPA”. “Cryoprotective solution”, “CPA”, “CPA solution” and “CPA cocktail” are used interchangeably herein.

“Vitrification CPA concentration” or “CPA concentration” as used herein relates to the concentration of the CPA(s) that are present in the CPA cocktail when the biological sample is cooled for vitrification. The vitrification CPA concentration can be determined to minimize injury to the biological sample during vitrification and rewarming. The vitrification CPA concentration is determined based on the CPA thermophysical behavior, expected cooling and rewarming conditions, and the CPA susceptibility of the biological sample.

“Cryomesh” as used herein relates to a porous surface/substrate that can retain a biological sample. The cryomesh can, for example, retain a biological sample on the filaments of the mesh while enabling the removal of at least some of the cryoprotective solution surrounding the biological sample. The cryomesh can, for example, retain the biological sample on the filaments of the mesh while enabling cooling, cryopreservation storage, and rewarming.

“Vitrification” as used herein relates to a biological sample that has attained a glassy, amorphous structure when cryopreserved at temperature below its glass transition temperature at a cryogenic temperature. Vitrified samples can be cryogenically stored at ultralow temperature (<−130° C.) in an ice-free glassy state. Vitrified samples may have less than 0.1% V/V of ice crystallization in the sample; however, vitrified samples may contain larger ice fractions if they may still produce a viable biological sample upon warming to superzero temperatures.

“Crystallized” or “frozen” sample as used herein relates to a biological sample that has attained some crystalline ice structure during cooling, storage, or rewarming and may not produce a viable biological sample upon warming to superzero temperatures. Crystallized samples may also be referred to herein as unvitrified samples, non-vitrified samples, or devitrified samples. These terms are used interchangeably herein.

“High-throughput” as used herein relates to the use of methods to rapidly process a large number of samples in a short amount of time.

“Critical cooling rate” or “CCR” as used herein relates to the minimum rate of temperature change required to cool a sample to a stable vitrified state without forming ice.

“Critical wanning rate” or “CWR” as referred to herein relates to the minimum rate of temperature rise needed to avoid ice crystal formation during rewarming of a vitrified sample.

The term “sub-millimeter” sample as referred to herein relates to a biological sample that is equal to or less than about a millimeter.

The term “millimeter” sample as referred to herein relates to a biological sample that is equal to or more than about a millimeter.

### SUMMARY

Aspects of this disclosure relate to methods for cryopreservation of precision-cut tissue slices comprising preparing one or more precision-cut tissue slices for cryopreservation by loading the one or more precision-cut tissue slices with a cryoprotective solution at a temperature lower than room temperature, and the cryoprotective solution comprising a permeable cryoprotective agent (CPA) and/or a nonpermeable CPA; transferring the one or more precision-cut tissue slices loaded with the cryoprotective solution to a porous substrate; and cooling the one or more precision-cut tissue slices on the porous substrate by submerging the precision-cut tissue slice and the cryomesh into a cryogenic coolant for vitrification of the one or more precision-cut tissue slices, wherein a cooling rate for vitrification of the one or more precision-cut tissue slices is equal to or greater than a critical cooling rate (CCR). For example, a minimum CCR is 24.6° C./min, whereas a preferred average cooling rate is greater than 7,000° C./min, 8,000° C./min, 9,000° C./min, 10,000° C./min or greater.

In one or more embodiments, the precision-cut tissue slices are from liver, kidney, lung, heart, brain, intestine or other human donor organs or tissues. In one or more embodiments, the precision-cut tissues slices are healthy or diseased liver tissue slices. In one or more embodiments, the tissue slices can be engineered tissue or micro physiological systems.

In one or more embodiments, tissue slice can be of a thickness of a sub-millimeter- or millimeter scale. In some embodiments, the term sub-millimeter- or millimeter scale slice can have a thickness of less than about ten millimeters (mm); or less than about five mm; or less than about one mm; or less than about 0.9 mm; or less than about 0.7 mm; or less than about 0.5 mm; or less than about 0.3 mm; or less than about 0.1 mm; or less than about 50 micrometers; or less than about 10 micrometer; or less than about 1 micrometer. In one or more embodiment, the tissue slice can have a thickness between about 50 micrometers and 500 micrometers.

In one or more embodiments, the loading of the one or more precision-cut tissue slices occurs at a temperature below room temperature. Such temperatures may be in the range of about −4° C. to about 12° C., for example, −4° C., 0° C., 4° C., 8° C., or 12° C.

In one or more embodiments, the precision-cut tissue slices are diffusively loaded with the cryoprotective solution.

In one or more embodiments, the cryoprotective agent is a penetrating cryoprotective agent. Penetrating cryoprotective agent can be, for example, ethylene glycol (EG), DMSO, propylene glycol (PG), methanol, glycerol, formamide, and the like. In one or more embodiments, the cryoprotective agent is a non-penetrating cryoprotective agent. Nonpenetrating cryoprotective agents can be, for example, sucrose, trehalose, lactose, sorbitol, Ficoll, polyethylene glycol (PEG), polyvinyl pyrrolidone (PVP), polyvinyl alcohol, polyglycerol, and the like. In one or more embodiments, the cryoprotective agent solution can include both penetrating and non-penetrating cryoprotective agents.

In one or more embodiments, loading of the cryoprotective agent is conducted in a stepwise manner. Step times can include loading steps greater than about 1 minute, greater than about 2 minutes, greater than about 3 minutes, greater than about 5 minutes, greater an about 5 minutes, greater than about 10 minutes, greater than about 15 minutes, greater than about 20 minutes, greater than about 30 minutes, greater than about 45 minutes, greater than about 60 minutes, or greater than about 90 minutes. In one or more embodiments, loading of the cryoprotective agent is conducted with ramp, logarithm, or exponential loading and the like.

In one or more embodiments, the porous substrate materials can include, for example, polymers, plastics, metals, nylon, carbon elastomer and the like. Plastics can include, for example, acrylics, polyesters, silicones, polyurethane, halogenated plastics, polyethylene, polypropylene, polystyrene, polyvinyl chloride, graphite, polydimethylsiloxane and the like. Mesh may include other natural and manmade polymers and all are within the scope of this description. Mesh may also include metals such as, for example, aluminum, copper, stainless steel and the like.

In one or more embodiments, the loading the one or more precision-cut tissue slices comprises loading the precision-cut tissue slices with the cryoprotective solution initially comprising about 10% ethylene glycol and increasing the concentration of ethylene glycol to a final loading concentration of about 40% ethylene glycol.

In one or more embodiments, the cryoprotective solution comprises 0.6M Sucrose in combination with ethylene glycol. The loading of the one or more precision-cut liver slices comprises increasing the ethylene glycol content of the cryoprotective solution in a step-wise manner, wherein in a first step the cryoprotective solution comprises about 10% ethylene glycol, a second step comprises about 25% ethylene glycol and a third step comprises about 40% ethylene glycol.

In one or more embodiments, the one or more tissue slices are vitrified in liquid nitrogen.

In one or more embodiments, the cryopreserved precision-cut tissue slices are provided for use in in-vitro pharmacological toxicity testing.

In one or more embodiments, the vitrified tissue slices can be stored for an extended periods of time meeting or exceeding 60 days and then rewarmed. In one or more embodiments, the precision-cut tissue slices may be stored for more than a day; or more than a weck; or more than a month; or more than 6 months; or more than a year; or more than 5 years.

In one or more embodiments, the one or more vitrified precision-cut tissue slices are rewarmed without ice formation. In one or more embodiments, the vitrified precision-cut tissue slices are rewarmed at a rate equal to or greater than the a critical warming rate (CWR). For example, a minimum CWR is 7703° C./min, whereas a preferred average warming rate is greater than 8,000° C./min, 9,000° C./min, 10,000° C./min or greater. In one or more embodiments, the rewarming comprises convectively rewarming the cryopreserved precision-cut tissue slices by immersing the vitrified precision-cut tissue slices in a rewarming solution. In one or more embodiments, the rewarming comprises other rewarming methods.

In one or more embodiments, the precision cut tissue slices are rewarmed after cryopreserved storage with maintenance of viability, function, tissue architecture and enzymatic activity comparable to controls. In one more embodiments, the precision cut tissue slices are convectively rewarmed by immersing the vitrified precision-cut liver slices in a rewarming solution comprising IM Sucrose at room temperature retains vitality of the slices.

In one or more embodiments the method further comprises unloading the cryoprotective solution from the precision-cut liver slices at 4° C. in one or more steps beginning with a solution comprising no ethylene glycol and 1M sucrose and reducing the concentration of the sucrose.

In one or more embodiments, the average pore size of the porous substrate is less than about one millimeter; or less than about 750 micrometers; or less than about 500 micrometers; or less than about 400 micrometers; or less than about 300 micrometers; or less than about 250 micrometers; or less than about 200 micrometers; or less than about 100 micrometers; or less than about 50 micrometers; or less than about 10 micrometers; or less than about 5 micrometers; or less than about 1 micrometers. In some embodiments, the average pore size can be greater than about one micrometer; or greater than about 10 micrometers; or greater than about 20 micrometers; or greater than about 30 micrometers; or greater than about 50 micrometers; or greater than about 75 micrometers; or greater than about 100 micrometers; or greater than about 250 micrometers; or greater than about 500 micrometers; or greater than about 750 micrometers; or greater than about 900 micrometers, or greater than about a millimeter.

In one or more embodiments, one or more tissue slices can be batch processed for cryopreservation. In one or more embodiments, this can include batch processing greater than about 6, or greater than about 24, or greater than about 48, or greater than about 96, or greater than about 500, or greater than about 1,000, or greater than about 2,000, or greater than about 5,000 tissue slices from a donor organ or tissue section.

Another aspect of this disclosure relates to a kit for vitrification of precision-cut tissue slices according to any one of the methods described herein, the kit comprising: a supply of cryoprotective solution according to one or more embodiments described herein; and a porous substrate for holding one or more precision-cut tissue slices during vitrification.

In one or more embodiments, the kit comprises premixed cryoprotective solutions; containers and fixtures to conduct cryoprotectant loading; a porous substrate for holding one or more precision-cut tissue slices during vitrification and storage; a wicking material; a container for storing the cryopreserved tissue slices on the porous substrate; and instructions for using the kit components for cryopreserving the tissue slices.

The instructions may include any one of the methods described herein alone or combinations thereof.

In one or more embodiments, the cryoprotective solution comprises a penetrating cryoprotective agent or a non-penetrating cryoprotective agent as described in one or more embodiments above.

In one or more embodiments, the porous substrate is a cryomesh and/or a porous substrate that is is an ETFE or nylon cryomesh and having a monofilament diameter of about 25 μm and about a 500 μm pore size.

In one or more embodiments, the kit comprises premixed solutions for unloading cryoprotectant the precision-cut tissue slices; containers and fixtures to conduct CPA unloading; and instructions for using the kit components for rewarming the tissue slices and unloading the cryoprotectant solution.

In one or more embodiments, the kit comprises a supply of 70% ethanol for initial cleaning and/or storage of the cryomesh and/or 10% tergazyme detergent for removing any precision-cut tissue slice tissue remnants remaining on the cryomesh after vitrification and rewarming.

Additional aspects of the disclosure relate to methods of culturing tissue slices using a cryomesh support and lowering a media height above tissue to a height sufficient to avoid supplemental external oxygenation of the media and maintaining maximum viability of the tissue slices, such that the media to tissue volume ration is 200:1 and the height of the media surface above the tissue slices is approximately 0.45 mm.

In one or more embodiments, the culturing is carried out after vitrifying the tissue slices for storage and transport of tissue slices in a vitrified state.

### DETAILED DESCRIPTION

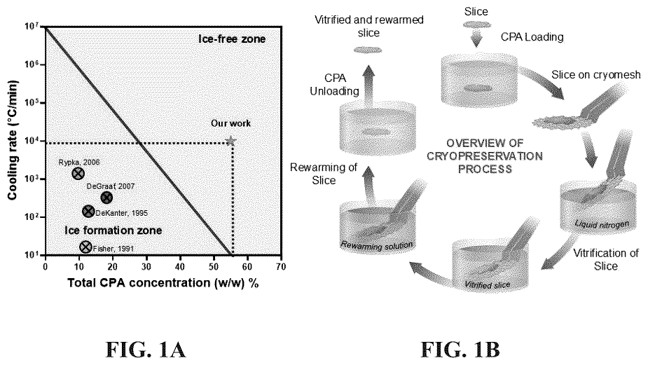

Methods and materials described herein illustrate uses of vitrification (an alternative to slow freezing), which uses higher concentrations of CPAs and high cooling rates to form a glass-like “ice-free” state. See FIG. 1A. Vitrification allows for the storage of Precision Cut Tissue Slices, PCTS, for example, Precision Cut Liver Slices (PCLS) at cryogenic temperatures with minimal to no loss in their viability, function, and enzymatic activity. To successfully vitrify, the PCTS must cool faster than the CCR (critical cooling rate required to avoid ice formation) and rewarm them faster than the CWR (critical rewarming rate required to avoid ice formation). Cryopreservation of PCTS by vitrification requires a delicate balance of having sufficient concentration of CPA to avoid ice formation under the achieved cooling and rewarming rates, while minimizing toxicity from the CPA.

High-throughput in vitro pharmacological toxicity testing is essential for drug discovery. For example, precision-cut liver slices (PCLS) provide a robust system for screening that is representative of the complex 3D structure of the whole liver. However, PCLS are not available as off-the-shelf products. With cryopreservation, slices can be shipped to laboratories without access to fresh tissue or used in planned experiments independent of surgical schedules. Aspects of this disclosure relate to an “ice-free” cryopreservation approach called vitrification and focus on culturing and assessing PCLS for 3 days post-vitrification and rewarming, given that most acute drug toxicity tests are conducted over 24 h to 72 h.

The cryopreserved PCLS according to methods described herein maintained high viability, morphology (tissue architecture), function, enzymatic activity, and drug toxicity response. Results show that the vitrified PCLS perform comparably to untreated controls and significantly outperform conventionally cryopreserved PCLS in all assessments (p<0.05).

Better understanding and predictability of the liver's role in specific drug metabolism and breakdown can help address the R&D challenges. Ideally, studying drug metabolism in the liver in vivo would be conducted, but this becomes prohibitive in terms of cost, accessibility of samples, and ethical concerns (Lake et al. 1993). Alternatively, an option exists to select and deploy properly characterized in vitro models such as precision cut liver slices (PCLS) as described herein. PCLS are powerful in that they closely resemble in-vivo architecture, cell populations and function. Since their introduction 30 years ago, PCLS studies have shown the ability to mimic drug pathways in humans thereby allowing improved prediction of in vivo drug efficacy and toxicity (Paish et al, 2019). PCLS can also be created from both diseased and non-diseased donor livers, making them excellent in vitro disease models for a wide range of biomedical research. Arguably the biggest barrier to PCLS use is the inability to deliver them in an “off the shelf” format because there are no standardized protocols for preserving PCLS like those that exist for cells. Improving protocols for preserving, storing and shipping PCLS would broaden access for their use in biomedical research. In addition, similar to organs, the functional viability of PCLS degrades rapidly over hours in cold storage. Under culture, PCLS can last for only a few days-their viability and function continue to decrease with time which could lead to inaccurate results and provides a limited practical window to conduct testing which occurs over years of development. To provide broad and timely access of PCLS for drug metabolism studies, we need an improved preservation approach that includes storage and standardized shipping.

Cryopreservation, which is the storage of biosystems at ultralow temperatures, could help solve this bottleneck. However, conventional cryopreservation approaches for PCLS using slow freezing in dilute concentrations of Dimethyl Sulfoxide (DMSO) (18-22 v/v %) have proven inadequate. While slow freezing (with ice) is commonly employed in cell suspensions, it fails in complex tissues where ice formation disrupts cellular and tissue structures, severely compromising the PCLS viability, morphology and function (de Graat et al., 2007). Indeed, the best PCLS freezing protocols published have resulted in only 70% viability after 4 hours in culture.

An alternative to slow freezing is vitrification which uses high concentrations of cryoprotective agents (CPAs) and rapid cooling rates to form a glass-like “ice free” state. Vitrification according to methods described herein would allow for the storage of PCLS at cryogenic temperatures with little to no loss in their viability, function, and enzymatic activity, for days, months and years of storage. An essential requirement for success is choosing an appropriate CPA to minimize toxicity and optimize loading and unloading protocols of the CPA to prevent osmotic injury prior to vitrification. As described herein, methods require a technology that can quickly cool the PCLS faster than the critical cooling rate (CCR, minimum cooling rate required to avoid ice formation) and rewarm the PCLS faster than the critical rewarming rate (CWR, minimum rewarming rate required to avoid ice formation) of the CPA.

In one embodiment, rat liver slices were diffusively loaded with a cryoprotective agent (CPA) cocktail consisting of Ethylene Glycol and Sucrose. The CPA-loaded PCLS were placed on a polymer cryomesh, vitrified in liquid nitrogen (LN2), and rapidly rewarmed in CPA. The vitrified and rewarmed PCLS were subsequently cultured in a of serum-free media for 3 days.

The cryopreserved PCLS maintained high viability, morphology, function, enzymatic activity, and drug toxicity response. Results show that the vitrified PCLS perform comparably to untreated controls and significantly outperform conventionally cryopreserved PCLS in all assessments (p<0.05).

While prior art does discuss the potential use of vitrification for PCLS, the previously presented work does not result in viability, function, tissue architecture and enzymatic activity comparable to controls, as presented in the current application. de Graaf et al. 2006, presents the methods utilized in the present application as the comparative “slow freezing and rapid thaw (FT)” control and notes that their separate attempts at vitrification of liver slices led to cryopreservation damage and worse functional outcomes than their FT protocol. Wishnies et al. 1991 also presents results for attempted vitrification of liver slices. While they show images of vitrified liver slices, they note that cloudy hazing was observed in the tissue during rewarming, which likely indicates that ice formation (devitrification) occurred during rewarming, which would result in similar damage to ice formation during slow cooling. Wishnies et al. 1991 presents limited functional data and their results relative to the controls is reflective of those observed for other attempts at freezing and thawing of liver slices (i.e. not liver slice vitrification, as in the present application).

### Methods

### Preparation of Precision Cut Liver Slices (PCLS)

In one embodiment, livers were recovered from 2.5-3-month-old Sprague-Dawley rats (Charles River Labs, Wilmington, MA) and sliced into 5 mm wide and 250 μm thick slices. All data are from three biologically replicate experiments (N=3) with 4 slices (n=4) per biological replicate for each experimental condition unless stated otherwise.

In further detail, in one or more embodiments, a 5 mm biopsy punch is used to obtain biopsies of the liver, followed by embedding of the biopsy onto the specimen tube using 3% (w/v) of low-melting agarose (IBI Scientific, IB70051) prepared in phosphate buffered saline (PBS). The specimen tube is then cooled using a chilling block to quickly set the agarose, followed by slicing using a Compresstome (Precisionary Instruments, MA, USA). The buffer tray of the Compresstome is filled with ice-cold PBS. The cut slices are collected in the tray, after which the agarose gel are removed, and the slices placed in cold modified UW solution until further use (2-3 hours).

### Culturing PCLS Using Mesh in 6 Well Plate

The slices were cultured in 6-well tissue culture plates with a of 1 ml of media per slice per well on nylon meshes with 0.3 mm filament thickness and 500 μm pore size under normoxic conditions in a 37° C. incubator. This maintained the media height above the tissue at 0.45 mm.

For example, the chemically defined culture media may consist of WilliamsE media (Gibco) supplemented with 10,000 ng/ml Insulin, 5.5 μg/ml Transferrin, 5 ng/ml Selenium (all Sigma), 2 mM L-glutamine (Sigma), 10 mM HEPES (Gibco), 50 μg/ml Gentamicin (Sigma), 2.5 μg/ml amphotericin-B (Sigma), and 0.1 μM Dexamethasone (Sigma). No serum is added to the culture media.

### CPA, Vitrification, and Rewarming Protocol

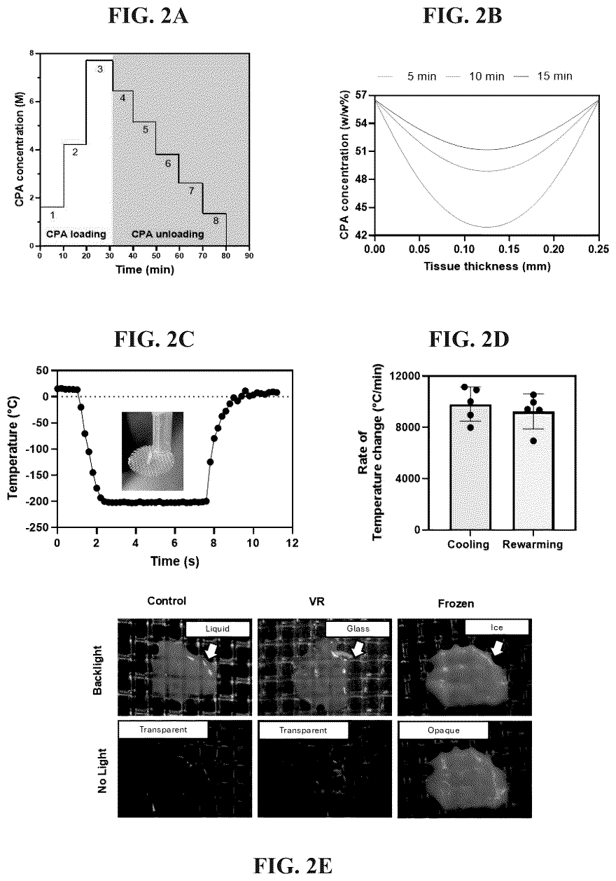

PCLS were transferred to ETFE cryomeshes with a monofilament diameter of 25 μm and 500 μm pore size. The PCLS were loaded with the CPA (diluted in modified UW carrier solution) in steps as follows 10% EG for the first step, 25% EG for the second step, followed by the final CPA concentration of 40% EG+0.6M Sucrose, as indicated in FIG. 2A. Once loaded with CPA, the PCLS were placed on the cryomesh and convectively vitrified by vertical immersion in liquid nitrogen to reduce Leidenfrost boiling as previously described. The PCLS were then convectively rewarmed by immersion in rewarming solution (1M Sucrose) at room temperature, followed by unloading the CPA from the slices at 4° C. in steps starting with (0 EG) and IM Sucrose followed by 0.7M, 0.525M, 0.35M, 0.175M Sucrose with the final step being the carrier solution as indicated in FIG. 2A.

In one or more embodiments, nylon meshes may be used for culturing of PCLS. For example, nylon meshes of 500 μm pore size and 0.3 mm filament thickness may be used for culturing. The meshes may initially be cleaned with 70% Ethanol. After use, the meshes may be washed in 10% Tergazyme detergent to remove any tissue remnants and then stored in 70% Ethanol under a bio safety cabinet.

The cooling and rewarming temperatures may be measured using a T-type fine gage bare wire thermocouple (COCO-002, OMEGA) and recorded using an oscilloscope (DSIM12, USB Instrument). Rates are then calculated from −100° C. to −40° C. and plotted as shown in FIG. 2D.

### COMSOL Model

COMSOL Multiphysics 5.6 (COMSOL, Burlington, MA) was used to model the CPA diffusion in PCLS. The preset properties for human liver were used for the model with heat capacity Cp=3540 J/(kg K), density=1079 kg/m3, thermal conductivity=0.52 W/(m K)¹⁹⁻²⁴. A 0.25 mm square geometry was used to represent the tissue, as the characteristic length of interest is the thickness of the tissue. The Transport of Concentrated Species physics model was used to estimate the diffusion of CPA components for loading times. A Mixture Average diffusion model was used, and the Maxwell-Stefan diffusivities for the CPA components were calculated using the formula reported by Yu et al²⁵ with the free diffusion coefficients of the components calculated using Stokes-Einstein formula. The species diffusivity was calculated as follows: EG-Sucrose→5.2260-12 m²/s, EG-carrier→3.85c-11 m²/s and Sucrose-carrier→5.618c-12 m²/s. The coefficient for Sucrose was calculated using the viscosity values provided by the National Bureau of Standards (Circular 440)²⁶ at 75 (% w/w) at 5° C. The temperature in the model was set to 277 K with a mixture density of 1140 kg/m³ and molar mass of EG, Sucrose and carrier to 0.062, 0.34 and 0.018 kg/mol respectively. An initial value of 4.2 M EG was set with 0 M Sucrose, and an inflow of 7.1 M EG and 0.6 M Sucrose was set as the boundary conditions on both ends of the tissue to estimate the final step time of CPA diffusion required for successful vitrification. The concentrations were then obtained for different timepoints of 5, 10, and 15 minutes. This is represented in FIG. 2B.

### Functional Assays and Cytochrome P4501A1 (CYP1A1) Live Tissue Imaging and Quantification

Assays for Urea, Albumin and ATP were performed according to manufacturer's instructions. For live imaging of CYP1A1 activity, the CYP1A1 was induced in the slices using 25 μM β-naphthaflavone for 24 h. PCLS were then incubated with 20 M 7-Ethoxyresorufin and 25 μM Dicumarol for 10 minutes, followed by imaging or quantification. The CYP1A1 cleaves the 7-Ethoxyresorufin to fluorescent resorufin that can be imaged and quantified²⁷. The slices were imaged using an excitation wavelength of 561nm laser in a Nikon A1RMP+ microscope. For quantification, the slices were placed in a microplate reader and imaged for 30 minutes in kinetic mode using 535/595 nm filters at 37° C.

Assay assessments. For example, assays were used for urea, albumin, and ATP as follows. For an assessment of urea, the slices are transferred to 24 well plates containing 0.25 ml of urea media for incubation times ranging from 1.5-3 hours. The urea media consists of a KHB base solution with 10 mM ammonium chloride (Sigma Aldrich) and 2 mM L-ornithine (Sigma Aldrich) to assist in the initiation of the urea cycle. The slices are then either snap frozen, stored, and then homogenized for ATP assessment or put back into culture with fresh culture media in incubators at 37° C. with 5% CO₂. For ATP, the slices were snap frozen and homogenized in 0.25 ml of sonication buffer solution containing 70% (v/v) of Ethanol and 2 mM EDTA (Ethylenediaminetetraacetic acid) at a pH of 10.9¹⁰. QuantiChrom Urea Assay Kit (BioAssay Systems) was used for the urea assay. Albumin was assessed from the culture media (1 ml) using an ELISA assay (Rat albumin ELISA Kit, ICL). Finally, Roche Bioluxminescence Assay Kit CLS II was used for ATP assay. All assays were performed according to the manufacturer's instructions.

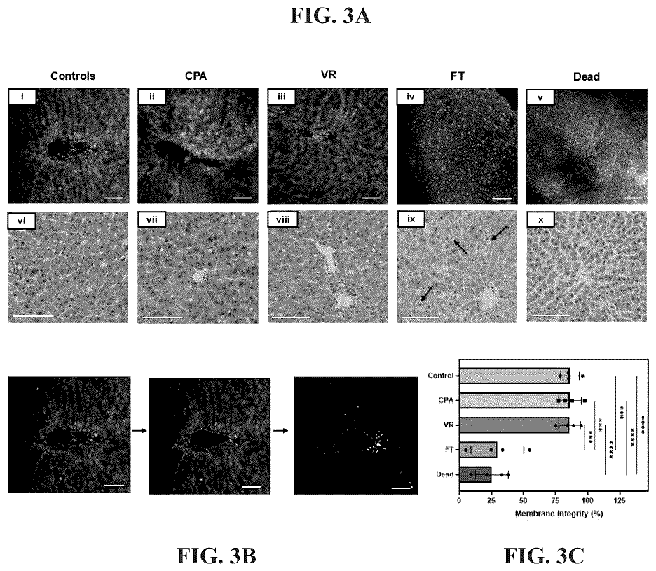

For example, for AO/PI live/dead images presented in FIG. 3A-3C, the PCLS were incubated with 8 ng/ml AO and 20 ng/ml PI (Millipore Sigma) for 5 min at room temperature. They were then imaged with an Olympus Fluoview 3000 inverted confocal microscope (Olympus) with 502/525-nm filters for AO and 493/636-nm filters for PI. The PCLS images were captured at 4,020×4,020-pixel resolution using a 20× magnification objective.

### Acetaminophen Drug Study with Cryopreserved Slices

Acetaminophen was prepared in culture media with supplements in increasing concentrations: 0, 1, 5, 10, 20, and 50 mM (see further below). Slices were then exposed to the APAP culture media for 24 hours, after which the media was replaced with media containing no APAP and cultured for 2 more days.

### Fixing, Embedding and Staining Procedure

PCLS were fixed in 10% Neutral Buffered Formalin. The slices were subsequently paraffin-embedded and 5 μm sections were then taken close to the center of the slices and stained for Hematoxylin & Eosin (H&E) and TUNEL.

### Total Protein Analysis

We also performed total protein analysis on the slices and found no significant difference between the protein amounts among the Control, CPA, VR, and FT groups. The Dead group shows lower protein, which can be explained by the loss of protein to the culture media during culturing due to extreme membrane damage and tissue degradation caused by freezing and thawing. Also note that the FT group could potentially have higher protein values than what is measured owing to similar damage caused by freezing and thawing. The protein content would also vary by slice since ice formation is uncontrolled during freezing and thawing.

### Image Analysis Using ImageJ

With ImageJ, a relative viability measure was obtained by counting the average number of dead cells (stained with red nuclei) to the total area by measuring the average area of each cell and allowing to estimate the number of cells in the given z-plane by excluding the central vein area and dividing the total area by the area of each cell. This was then followed by converting the red channel (PI signal) to 8-bit, followed by thresholding and counting the number of red nuclei using particle analysis, setting a threshold of 10 pixel² area. The membrane integrity is presented as a percent using this image analysis method in FIG. 3C.

### CYP1A1 (Resorufin Production) Live Imaging and Quantification

For live imaging of CYP1A1 activity, the slices were incubated with culture media containing 25 M B-naphthaflavone made in DMSO with 2.1 (v/v) % final DMSO concentration) for 24 hours to induce CYP1A1. They were then incubated with WilliamE media (no phenol red), 20 μM 7-Ethoxyresorufin, and 25 μM Dicumarol for 10 minutes and imaged using an excitation wavelength of 561 nm laser in a Nikon A1RMP+ microscope. The CYP1A1 cleaves the 7-Ethoxyresorufin to fluorescent resorufin that can be imaged and quantified²². For quantification, the slices were placed in a microplate reader (Synergy HT, BioTek) and imaged for 30 minutes in kinetic mode using 535/595 nm filters at 37° C.

Reagents and Equipment are shown in TABLE 1 below.

TABLE 1

| Equipment/Reagent | Company | Location |
|---|---|---|
| Compresstome | Precisionary | Ashland, Massachusetts, |
| Model VZ-310-0Z | Instruments | USA |
| β-naphthaflavone | Cayman Chemical | Ann Arbor, Michigan, USA |
| 7-Ethoxyresorufin | Company | |
| Dicumarol | | |
| Acetaminophen | | |
| ETFE Cryomesh | Electron Microscopy | Hatfield, Pennsylvania, |
| Cat# 64700-24 | Sciences | USA |
| Nylon culturing | McMaster Carr | Elmhurst, Illinois, USA |
| mesh Cat #9318T44 | | |
| Microplate reader | BioTek | Winooski, Vermont, USA |
| Synergy HT | | |

### Statistics

Statistical analysis was performed in R version 4.3.2 (R Foundation for Statistical Computing, Vienna, Austria). Normality was established using the Shapiro-Wilk test to compare continuous variables, and homogeneity of variance was assessed using Levene's test. For normally distributed group comparisons, ANOVA testing with pairwise post hoc t-test for single comparisons or Tukey HSD test for multiple comparisons were used. Non-normal variables were tested using the non-parametric Kruskal-Wallis and pairwise Wilcox (Mann Whitney U) or Dunn's tests for individual group comparison. Grubb's test and IQR test were used to screen and censor outlier data. The Benjamini-Hochberg method was used to adjust for multiple comparisons. A p-value less than 0.05 was taken to be statistically significant (*) (p<0.005 represented by **, p<0.0005 by ***, p<0.00005 by ****). Continuous data are presented as mean±standard deviation. Only statistically significant differences are shown in the figures.

### Results

### Vitrification and Rewarming of PCLS

This embodiment tested cryopreservation and post-rewarming viability, function, enzymatic activity, and drug response in 250 μm thick×5 mm diameter PCLS from Sprague Dawley rats as a model system. The liver slices using a cryoprotectant cocktail consisting of 40% (v/v) of EG and 0.6M Sucrose, were initially developed for the cryopreservation of hepatocytes (Magalhães et al., 2008), which we have used in whole rat livers (Sharma et al., 2021). A modified version of University of Wisconsin (UW) solution as carrier may be used (starch-free with a 1 g/L concentration of PEG35 k for oncotic support, as previously reported).

Osmotic injury and toxicity are two of the main challenges to resolve when designing CPA loading and unloading protocols. For this study, CPA loading and unloading were performed stepwise to limit osmotic injury (FIG. 2A). CPA exposure was conducted at 4° C. to reduce toxicity, as we previously used in rat livers (Sharma, 2021).

The CPA loading protocol was determined by developing a multicomponent diffusion model in COMSOL that estimated EG and Sucrose tissue concentration over time, as previously described for other permeant CPAs (Yu et al., 2022). The model tested three step durations (5, 10, and 15 minutes). At 5 minutes, the model predicted a minimum CPA concentration of 42.8 (% w/w) in the interior of the PCLS, whereas this increased to 48.8 (% w/w) at 10 minutes. Aa 10-minute step duration was chosen for this specific CPA loading and unloading process to reach a minimum of 48 (% w/w) of CPA.

The application herein describes specific and generalized methods for cryopreservation precision-cut tissue slices. As illustrated in FIG. 8, PCLS were additionally exposed to alternative vitrifiable CPA formulations, following the same CPA loading and unloading protocol as described above. The PCLS maintained high viability after exposure to the modified cryoprotectants. The specifications described herein can be used to modify the current protocol based on reasonable experimentation, to identify an appropriate CPA formulation and CPA loading protocol, based on the tissue type and tissue thickness, to effectively vitrify and rewarm a variety of tissue slices.

The vitrification and rewarming procedure were then experimentally validated by thermometry and visual inspection (n=5). Vitrification of the slices with 10-minute loading steps indicated no visible ice (FIG. 2E).

The vitrification and rewarming of the PCLS were done on ETFE cryomeshes. Temperature measurements taken during cooling and rewarming of the slices indicated average cooling rates of 9,800° C./min and rewarming rates of 9,200° C./min (FIG. 2C, 2D), which exceeded the expected CCR (24.6° C./min) and CWR (7703° C./min) at the minimum required concentration (48 w/w %) calculated from previous studies¹¹.

### Viability and Morphology of Cryopreserved PCLS

The viability of the slices was assessed using AO/PI viability stain (live cells are green in color, and dead cells are red). Fresh control groups were compared to PCLS treated with CPA-only (CPA loaded and unloaded), vitrification and rewarming (VR), conventional cryopreservation (slow freczing and rapid thawing in cryovial after loading with 18% DMSO (v/v) for 30 minutes on ice, FT), and a negative control group (freeze-thawed 3 times with no CPA, Dead) (FIG. 3A). Live and dead cell fractions were quantified by image analysis (FIG. 3B. 3C).

The morphology of the slices was assessed by histologic staining (H&E), which showed that PCLS from the Control, CPA, and VR groups had well-preserved hepatocytes, microarchitecture, and portal tracts. In comparison, the FT group exhibited significant vacuolization (marked with arrows in FIG. 3A ix), and the Dead group showed significantly damaged microarchitecture. These were predicted to substantially affect viability, consistent with the measures observed.

### Culture Conditions for Liver Slices

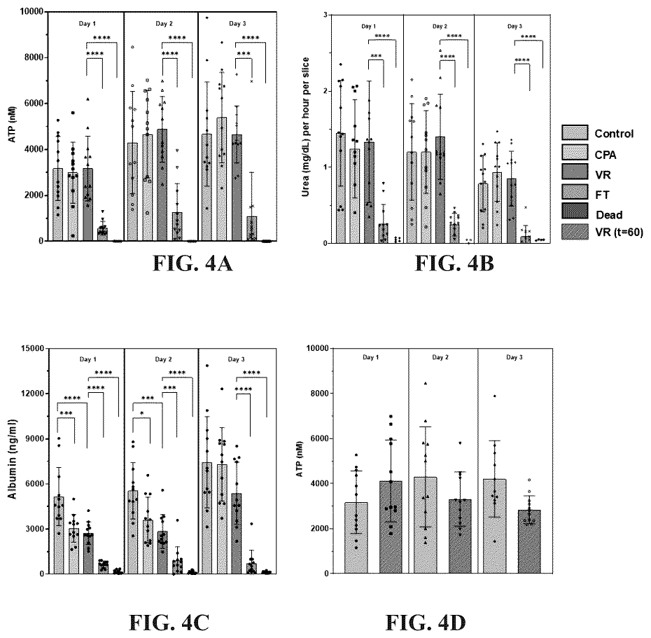

Previous attempts have been made to culture liver slices for extended periods, but for most drug discovery and testing applications, immediate acute toxicity tests are conducted within 24 hours. When conducting trials for daily dose drugs, there are perspectives on how an optimal drug design for oral drugs should aim to have a half-life of 12-48 h. So, we set culturing timeframes to be 3 days for the initial development of the study as a conservative estimate for the practical window needed for drug studies. Previous studies have shown that sufficient passive oxygenation for hepatocytes can be provided by adjusting the media height above the cells, eliminating the need for high levels of external oxygenation. Therefore, we systematically studied the height of the media above the PCLS, striving to avoid supplemental external oxygenation of the media whilst maintaining maximum viability and function. We found that 1 ml of media was required to sustain a single slice over 24 hours (200:1 media to tissue volume) and optimized the media height above the tissue to 0.45 mm. Additionally, culturing the slice on meshes enhanced oxygen diffusion from both sides. The controls slices cultured directly on the meshes also did not show any difference in viability, function or morphology compared to the VR group (FIG. 3, FIG. 4) that was cryopreserved on ETFE meshes and then transferred to nylon meshes to culture. This resulted in our ability to culture PCLS with high viability, function, and drug response for up to 3 days in culture.

### Metabolic Health and Functional Assessments in Cryopreserved Slices

We assessed the metabolic health of the PCLS by their ATP levels (FIG. 4A). The ATP levels of the controls, CPA, and VR groups remained high and consistent throughout the 3 days in culture (FIG. 4A). The FT group showed significantly lower ATP levels throughout the 3-day culture period, while the Dead group remained below the detection limit of the assay used (<25 nM).

Good function of the cryopreserved slices is required for effective biomedical and drug testing applications. To measure liver slice function, we analyzed urea production (FIG. 4B) and albumin synthesis (FIG. 4C). With urca production, the CPA-only and VR groups behaved similarly to Controls, where urea production remained consistent throughout the 3 days in culture with mean values equal to or above 0.7 mg/dL for all three groups. A slight drop in urca production was observed on day 3 but was not statistically significant. The FT group showed significantly less urca production over all 3 days, consistent with other measurements. The dead group showed no measurable urca production.

Albumin synthesis stayed high and consistent with the control groups over all three days (FIG. 4C). The CPA and VR groups experienced a statistically significant initial decrease in albumin synthesis compared to controls but then caught up to controls on the third day of culture. This could be due to recovery of the slices from an initial stress response on exposure to the CPA and during vitrification/rewarming, but it did not seem to hinder the ability of the slices to synthesize new albumin³². An increase in albumin synthesis was seen with the controls, CPA, and VR groups when comparing day 1 to day 3, indicating preserved function of the CPA and VR slices. The FT and dead groups had significantly less albumin synthesis over all 3 days in culture. However, the dead controls still showed low albumin levels, possibly indicating a slow leakage of pre-formed albumin rather than actual synthesis.

To demonstrate that the slices could be stored for extended periods of time once vitrified, we stored vitrified slices at −150° C. for 60 days, rewarmed the slices, unloaded the CPA, and assessed their ATP levels (FIG. 4D). The ATP levels of the stored slices were consistent across the three days in culture and in comparison to Controls, the stored and rewarmed slices (t=60) did not show any differences in ATP on all three days of culture. Vitrification has been shown to maintain a stable cryopreserved state over extended periods of time (theoretically capable of indefinite storage), allowing for potential stable, cryopreserved storage for days, months, and years.

### Acute Testing for Zonated CYP Activity, Viability and Apoptosis

Liver slices afford the opportunity to assess the zonal activity of cytochrome P-450 (CYP) enzymes that form the crux of xenobiotic metabolism. These enzymes are zonated, with the most activity present in zone 3 near the central hepatic vein draining the hepatic lobule. To understand if the zonal CYP activity is observed in the VR slices, we induced CYP1A1 in the control and VR PCLS for 24 h, followed by imaging. This allowed for assessment of zonal CYP activity after cryopreservation where the tissue may undergo significant change and injury. We also quantified the activity throughout the 3 days in culture. We chose to compare the CYP activity only between the Controls and the VR groups as we posit that since even basic function is severely impaired in the FT and Dead groups, that enzymatic activity, apoptosis and drug response would also be of significantly lower performance.

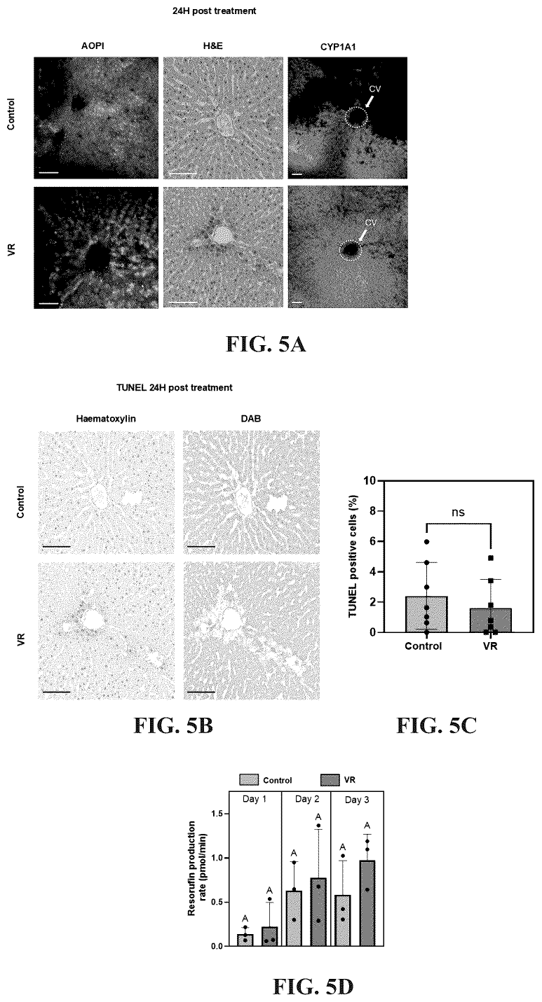

By confocal imaging, the cleaved resorufin was visible as a bright signal near the central vein in the acini of the liver slices in both the Control and VR groups, indicating zonated CYP activity in zone 3. Quantification of fluorescence demonstrated that resorufin production rate was induced and similar between control and VR PCLS over the 3-day culture. Both assessments showed that CYP activity post cryopreservation was zonated in the acute 24-hour period and that activity was maintained during all 3 days in culture (FIG. 5A, 5D).

TUNEL staining was also performed after 24 h to assess induction of apoptosis (or necrosis) (n=7). The TUNEL-positive cell fraction did not differ between VR and control PCLS (FIG. 5B, 5C). Control and VR groups showed an average of 2.8% and 1.8% TUNEL-positive cells, respectively, which was not statistically different.

### Acetaminophen (APAP) Hepatotoxicity in Fresh and Cryopreserved PCLS

The most critical issue related to in vitro drug studies is the potential for hepatotoxicity, representing a major failure mechanism for pharmaceuticals. To demonstrate the use of cryopreserved PCLS as an in-vitro model for drug testing, we exposed the control, VR, and Dead slices to different concentrations of APAP. We chose APAP as a model drug for testing since it is well-studied, and APAP overdose is a leading cause of hepatotoxicity, which in some cases could lead to fatal acute liver failure³³.

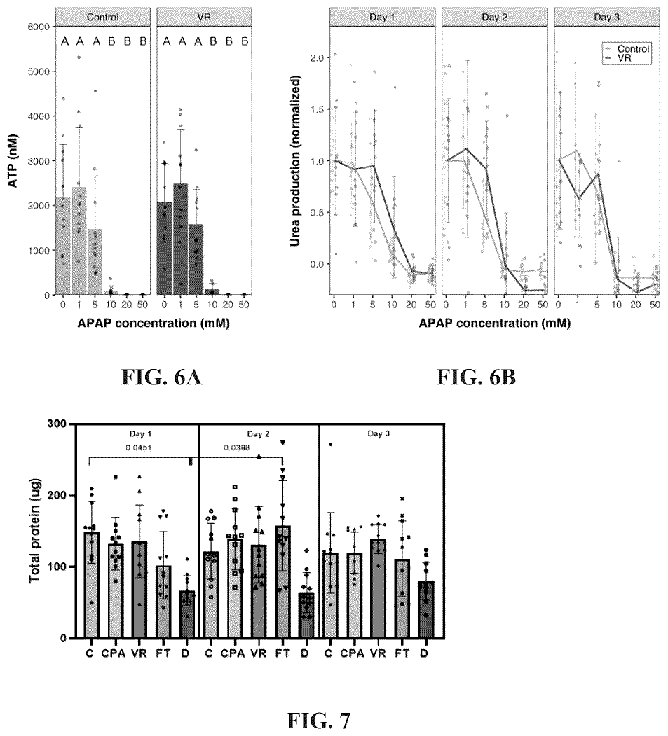

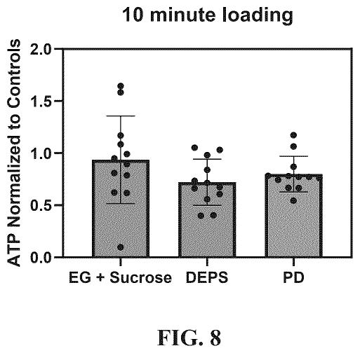

Liver slices were exposed to increasing concentrations of APAP for 24 hours. The APAP dosage was chosen based on previous studies in liver cell lines. Urea was measured in the same slices over 3 days in culture (FIG. 6B). The slices were homogenized at the end of 72 hours to measure ATP content (FIG. 6A). VR and Control slices performed very similarly, maintaining function and viability APAP concentrations to 5 mM, but 10 mM APAP resulted in severe injury and loss of function that increased over the culture period. At 20 mM and 50 mM APAP exposure, complete necrosis of slices occurred even on day 1, with no urea detectable. The ATP content in the slices at the end of day 3 also showed similar trends, with the slices exposed to 0-5 mM APAP maintaining ATP content while the slices exposed to 10-50 mM APAP showed undetectable ATP levels (below 25 nM). The ATP levels at Day 3 for the APAP study are lower than that shown in FIG. 4A. The study in FIG. 4A had dedicated slice groups for each measurement day, while the same set of slices were measured daily over three days in culture for the APAP study, requiring daily exposure to ammonium chloride. We posit that repeated exposure of the slices to high concentrations of ammonium chloride (10 mM) over the course of 3 days in culture led to some loss in viability.

### Discussion

Preventing drug failures during human clinical trials requires careful evaluation during the pre-clinical stage. This capability can prevent patient harm, eliminate ineffective therapies, and ultimately increase successful outcomes while lowering costs. In the absence of in vivo testing, which is cost-prohibitive and not fully accessible, cryopreserved liver tissue slices are a promising solution. PCLS banking from representative patient populations would open the possibility for repeatable drug screening and provide an accurate and patient-representative model for studying diseases and their progressions. New drug candidates and treatments could be tested on identical representative patient populations over the course of months and years using banked PCLS. PCLS banking would be facilitated by placing the cryopreserved PCLS on the mesh, in sealed, sterile storage containers, where they could be kept for days, weeks, months, or years. Storage containers could be inventoried and also shipped to other locations for rewarming. In this study, we illustrated in detail the first successful cryopreservation of rat liver slices through ice-free vitrification and rewarming to maintain viability, function, and enzymatic activity and show drug response post-rewarming out to 3 days.

As mentioned, we focused on the culture of liver slices for 3 days, an acute testing window for a wide range of biomedical applications. Notably, no current standardized methodology for the culturing of liver slices exists. Many culturing methods involve using large Erlenmeyer flasks and culturing the slices in external oxygenated environments using carboxygen. Even with continuous improvement in culturing technology, the viability with most PCLS culture has only been reported for 24-48 h (Olinga et al., 2001, Granitzny et al., 2017). The most extended culture of PCLS were reported by Parish et al., who cultured PCLS for 6 days using a bioreactor, Jagatia et al, who cultured them for 8 days using an orbital shaker and Wu et al., who reported culturing PCLS for 15 days. However, the latter reported a decrease in specific gene expressions over the days in culture, which may indicate a transition to a non-representative sample. Inspired by air-interface culturing systems, we achieved a reproducible culture of liver slices for up to 3 days using the simple technique of suspending the PCLS on nylon meshes-this and optimizing the media height above the tissues allowed for better oxygen diffusion into the tissue. In comparison to literature, we report ATP and albumin values that are consistent with viable and functional PCLS over days in culture.

Having established a culture approach that can be reliably replicated, we turned to cryopreservation. Unsuccessful previous studies on liver slices employed slow rates of freezing and thawing (<2,000° C./min cooling and rewarming) in cryovials using low concentrations of DMSO (18-22% v/v). These methods resulted in poor viability and function of slices even after short-term culture. The poor viability could be attributed to disruption of the cell membranes due to ice formation and damage to the necessary extracellular microarchitecture disrupting normal function. Another major challenge with conventional methods for PCLS cryopreservation is a lack of reproducibility, given the limited control over where ice formation occurs. Vitrification circumvents these issues and allows for preserving PCLS for indefinite periods. Until now, limited attempts at vitrification and rewarming liver slices have been reported (Wishnies et al., 1991), but the maintenance of viability and function of tissues over multiple days in culture has not been demonstrated.

Selecting the right CPA and protocol for loading and unloading were critical to succeed at vitrification and rewarming, as injury can manifest in the form of osmotic injury and direct CPA toxicity. Osmotic injury occurs when the cells are exposed to sizeable transmembrane concentration gradients of the CPA, which causes acute shrinking of cells during loading and swelling of cells during unloading, leading to injury. One of the mechanisms of toxicity is the metabolization of the CPA components by the cells and their conversion into toxic byproducts. Osmotic injury can be minimized by controlled loading and unloading of the CPA, mitigating acute shrink-swell behavior. We reduced toxicity by lowering the temperature of CPA exposure, which slows the metabolism of the components by the cells. Hence, we loaded the PCLS stepwise at 4° C. to minimize injury and vitrified and rewarmed slices at high cooling and rewarming rates using a cryomesh. The VR slices demonstrated markers comparable to fresh slice controls for all metrics assessed in this study. This observed behavior was consistent for the VR slices over 3 days in culture. Slices stored for 60 days and rewarmed also show consistent function over 3 days in culture establishing our method as one that can enable “off-the-shelf” shipping in the future

One of the critical differences that the PCLS provide as in-vitro tools over cells is their ability to capture zonal activity during xenobiotic liver function. In this study, CYP1A1 activity was similar and sustained for control and VR PCLS for all days in culture. This has important implications for drug testing protocols and understanding the progression of liver diseases. The initiation and progression of liver diseases also occur in a zone-wise manner, with many non-alcoholic and alcoholic diseases of the liver beginning in zone 3 and progressing to zone 1. These zone-dependent disease and signaling pathways cannot be studied using conventional cell-based in-vitro models.

The use of VR PCLS for drug hepatoxicity was demonstrated with the common toxin, APAP, with demonstration of dose-dependent toxicity similar to control PCLS. The response of the VR group to APAP was similar to Controls, indicating no significant differences between the two groups on a macroscopic level over 3 days of culture. Most prior APAP toxicity testing outlined in the literature was performed using liver cell models. Increased toxicity levels have been reported at primarily high concentrations (>10 mM), but injury also occurs at lower concentrations (≤5 mM), where mitochondrial injury is thought to be the leading cause. In this PCLS study, there was no significant difference in ATP levels on APAP exposure from 0 mM to 5 mM in both groups. This indicates the opportunity to research the mechanisms of APAP toxicity using PCLS, which would more closely resemble in-vivo whole liver conditions.

To summarize, this study used rat liver slices as a model system to establish successful vitrification and rewarming using the cryomesh. These methods can be used in the obtaining of human tissue to demonstrate success with both normal and diseased human liver slices. Overall, the methods described herein can expand the use of PCLS in pharmacology and hepatology.

Cryopreservation of PCLS through vitrification may allow for the development of an “off-the-shelf” cryo supply chain of human PCLS that are stored in a repository and available for on-demand shipping for research. The translation of this technology may allow PCLS to become a scalable, reproducible, wide-ranging, and population-representative source of tissue that can accurately mimic in-vivo conditions of the human liver thereby revolutionizing our methods of drug discovery and development.

The rat livers used in this study could easily be processed to produce 150-200 of the 5 mm diameter PCLS per liver. Human donor livers or liver sections will be larger than a rat liver and can yield a greater number of slices, so the limitation for the currently described PCLS cryopreservation will be throughput for vitrification. As previously described (Guo et al. Advanced Science 2024) cryomesh based vitrification approaches can be scaled with increases in the mesh size. It is anticipated that once rewarmed, PCLS will be utilized in existing testing and culture workflows, which utilize e.g. 6-, 12-, 24-, 48-, or 96-well plate formats. It is anticipated that PCLS could be vitrified in batches compatible for use with these culture and testing formats. For example, 96 PCLS could be vitrified on a mesh roughly at least a size of 6 cm×4 cm (larger to accommodate spacing between the slices). After slicing, large quantities of PCLS could be CPA loaded in bulk solution and then rapidly placed on a mesh prior to vitrification or (ii) distributed on a mesh, then CPA loading by moving the mesh between reservoirs for different CPA loading steps or exposing and removing the CPA on the mesh for each step. Estimating that donor liver tissue will remain viable in cold storage for 8-12 hours, and that a single technician could process roughly one large mesh per hour, throughput for this case could be up to roughly 1,000 PCLS vitrified. Considering that multiple technicians and/or multiple mesh could be processing in parallel, throughput for the current PCLS methods could be up to 5,000 or potentially up to 10,000 slices per donor liver.

What is claimed:

1. A method for cryopreservation of precision-cut tissue slices comprising:
preparing one or more precision-cut tissue slices for cryopreservation by loading the one or more precision-cut tissue slices with a cryoprotective solution at a temperature lower than room temperature, and the cryoprotective solution comprising a cryoprotective agent (CPA) solution;
transferring the one or more precision-cut tissue slices loaded with the cryoprotective solution to a porous substrate; and
cooling the one or more precision-cut tissue slices on the cryomesh by submerging the precision-cut tissue slice and the cryomesh into a cryogenic coolant for vitrification of the one or more precision-cut tissue slices,
wherein a cooling rate for vitrification of the one or more precision-cut tissue slices is equal to or greater than about 25° C./min.

2. The method of claim 1, wherein the tissue is healthy or diseased liver tissue.

3. The method of claim 1, wherein the loading of the one or more precision-cut tissue slices occurs at a temperature in the range of about −4° C. to about 12° C. and/or the precision-cut tissue slices are diffusively loaded with the cryoprotective solution.

4. The method of claim 1, wherein the cryoprotective agent is at least one penetrating cryoprotective agent, including ethylene glycol, ethylene glycol, DMSO, propylene glycol (PG), methanol, glycerol, or formamide.

5. The method of claim 1, wherein the cryoprotective agent also includes at least one non-penetrating cryoprotectant, including sucrose, trehalose, lactose, sorbitol, Ficoll, polyethylene glycol (PEG), polyvinyl pyrrolidone (PVP), polyvinyl alcohol, or polyglycerol.

6. The method of claim 1, wherein loading the one or more precision-cut tissue slices comprises loading the precision-cut tissue slices with the cryoprotective solution initially comprising about 10% ethylene glycol and increasing the concentration of ethylene glycol to a final loading concentration of about 40% ethylene glycol and optionally the cryoprotective solution comprises 0.6M Sucrose in combination with ethylene glycol and optionally wherein the loading of the one or more precision-cut tissue slices comprises increasing the ethylene glycol content of the cryoprotective solution in a step-wise manner, wherein in a first step the cryoprotective solution comprises about 10% ethylene glycol, a second step comprises about 25% ethylene glycol and a third step comprises about 40% ethylene glycol.

7. The method of claim 1 and further comprising storing the vitrified tissue slices for an extended periods of time meeting or exceeding 60 days.

8. The method of claim 1, wherein the tissue slices are rewarmed at an average rewarming of at least about 9,000° C./min and without ice formation.

9. The method of claim 1 and further comprising convectively rewarming the cryopreserved precision-cut tissue slices by immersing the vitrified precision-cut tissue slices in a rewarming solution comprising 1M Sucrose at room temperature.

10. The method of claim 9 and further comprising unloading the cryoprotective solution from the precision-cut tissue slices at a temperature in the range of about −4° C. to about 12° C. in one or more steps beginning with a solution comprising no ethylene glycol and 1M sucrose and reducing the concentration of the sucrose.

11. The method of claim 1, wherein the tissue slices maintain comparable viability, morphology and architecture, and function comparable to fresh control tissue slices, after vitrification and rewarming.

12. The method of claim 11, wherein the cryopreserved precision-cut tissue slices are provided for use in in-vitro pharmacological toxicity testing.

13. A kit for vitrification of precision-cut tissue slices according to the method of claim 1, the kit comprising:
a premixed cryoprotective solution;
containers and fixtures to conduct cryoprotectant loading;
a porous substrate for holding one or more precision-cut tissue slices during vitrification;
a wicking material;
a container for storing the cryopreserved tissue slices on the porous substrate; and
instructions for using the kit components for cryopreserving the tissue slices.

14. The kit of claim 13, wherein the cryoprotective agent is at least one penetrating cryoprotective agent, including ethylene glycol, ethylene glycol, DMSO, propylene glycol (PG), methanol, glycerol, or formamide and/or includes at least one non-penetrating cryoprotectant, including sucrose, trehalose, lactose, sorbitol, Ficoll, polyethylene glycol (PEG), polyvinyl pyrrolidone (PVP), polyvinyl alcohol, or polyglycerol.

15. The kit of claim 13, wherein the storage container enables long-term, sealed, sterile storage of the tissue slices and/or wherein the storage container can be integrated into an inventory system for long-term storage or a shipping container for shipping and transport, wherein optionally the container allows storage for at least 60 days.

16. The kit of claim 13, wherein the porous substrate is configured to allow cryopreservation of multiple tissue slices, such as 6, 12, 24, or 96 tissue slices on a single porous substrate and optionally the kit includes multiple porous substrates to allow cryopreservation of larger numbers of tissue slices, and where multiple kits could be used to vitrify up to 10,000 tissue slices per donor organ or tissue section.

17. A kit for rewarming cryopreserved tissue slices according to the method of claim 1, the kit comprising:
premixed solutions for unloading cryoprotectant from the tissue slices;
containers and fixtures to conduct CPA unloading; and
instructions for using the kit components for rewarming the tissue slices and unloading the cryoprotectant from the tissue slices.

18. The kit of claim 17, the premixed solutions are used for rewarming the tissue slices wherein one or both of rewarming is conducted without ice formation and the tissue slices maintain comparable viability, morphology and architecture, and function comparable to fresh control tissue slices, after vitrification and rewarming.

19. A method of culturing tissue slices using a cryomesh support and lowering a media height above tissue to a height sufficient to avoid supplemental external oxygenation of the media and maintaining maximum viability of the tissue slices, such that the media to tissue volume ration is 200:1 and the height of the media surface above the tissue slices is approximately 0.45 mm.

20. The method of claim 19 and further comprising vitrifying the tissue slices for storage and transport of tissue slices in a vitrified state.
